(19) United States
(12) Patent Application Publication
(10) Pub. No.: US 2025/0017580 A1
(43) Pub. Date: Jan. 16, 2025

(54) MODIFICATIONS TO ACCESS PORTS FOR MINIMALLY INVASIVE NEURO SURGERY

(71) Applicants: Robert Trimarche, Lynchburg, VA (US); Dilantha B. Ellegala, Lynchburg, VA (US)

(72) Inventors: Robert Trimarche, Lynchburg, VA (US); Dilantha B. Ellegala, Lynchburg, VA (US)

(21) Appl. No.: 18/901,838

(22) Filed: Sep. 30, 2024

Related U.S. Application Data
(63) Continuation of application No. 17/190,806, filed on Mar. 3, 2021, now abandoned.
Application No. 17/190,806 is a continuation of application No. 15/328,175, filed on Jan. 23, 2017, now abandoned.
(60) Provisional application No. 62/028,023, filed on Jul. 23, 2014.

(51) Int. Cl. A61B 17/02; A61B 5/00; A61B 17/34; A61B 34/20; A61B 90/57

(52) U.S. Cl. CPC A61B 17/0218; A61B 5/0086; A61B 5/4064; A61B 5/407; A61B 17/3417; A61B 17/3439; A61B 17/3494; A61B 17/3496; A61B 34/20; A61B 2034/2055; A61B 2034/2068; A61B 2090/571

(57) ABSTRACT

An access port or retractor tube provides access through tissue to a surgical site or field, such as at the brain or spine, in a minimally invasive manner. The access port permits a user to clearly view and access the surgical field, including areas medial thereto, in a minimally invasive manner by dilating of separating tissue rather than cutting tissue. Neuro monitoring and neuro navigation are tools essential to neuro surgery to protect vital and eloquent tissues. Combining navigation and monitoring into the access ports/reactor tubes would enable the surgeon to be more precise and efficient during minimally invasive procedures while still being maximally effective in protecting non operative tissues.

### CROSS-REFERENCE TO RELATED APPLICATION

This is a Continuation of U.S. application Ser. No. 17/190,806 filed on Mar. 3, 2021, which is a Continuation Application of U.S. application Ser. No. 15/328,175 filed on Jan. 23, 2017, claiming priority from U.S. Provisional Application No. 62/028,023 filed Jul. 23, 2014 the disclosure of which is incorporated herein by reference.

### TECHNICAL FIELD

The present invention(s) relate to neuro surgery equipment, and more particularly, to access ports, retractor tubes, locator rods and sensors for neuro monitoring and neuro navigation.

The present invention(s) relate to methods and devices for minimally invasive brain and spine surgery and devices for performing said surgeries. More specifically, the current invention(s) are modifications to existing minimally invasive access retractor ports and locator/dilating tubes with integration of neuro navigation/neuro monitoring.

### BACKGROUND ART

In the field of neuro surgery, including brain and spine surgery, it is extremely important to be able to monitor the integrity of neural tissue during surgery. Neuro monitoring is a procedure in which the electrical conductivity of peripheral nerves and control centers in the brain are monitored with real time feedback given to the surgeon while operating, to allow him/her to know if there has been any compromise to eloquent tissues that control motor and or sensory function, thereby reducing risk of adverse events such as paralysis, pain or numbness.

Neurosurgeons routinely use neuro navigation during surgery as well. Navigation is a computer system that integrates pre-operative scans such as CT or MRI with the patient's actual anatomy in the operating room, allowing the surgeon to know where he/she is in the brain or spinal cord. This enables the surgeon to steer clear of very sensitive nervous system tissues, while performing any number of required procedures, such as brain/spine tumor resections, aneurysm clippings and pedicle screw placement during spinal fusions. This technology has had advancements in the last several years, combining the scan images into the operating microscope, reducing the surgeons' time of coming out of the operative field to reassess the exact location of the anatomy in question.

### DISCLOSURE

### Technical Problem

Over the last decade, an effort has been made in the field of surgery to perform operations in a minimally invasive setting. Medical literature shows that with minimally invasive approaches, the patient's recovery is faster and hospitalization is shorter. With minimally invasive surgery, techniques and tools have been developed to reduce trauma to non-essential tissue; however, increasing the final diameter of these tools may cause undesired damage to a patient's tissue.

### Solution to Problem

In accordance with an aspect of the present invention(s), there are several design modifications to access retractor ports, as well as the tools for placing said ports into the patient such as incorporation of a gasket. The inventions are also intended to combine neuro navigation and neuro monitoring into the retractor ports and locator rods thereby increasing the ability to perform minimally invasive surgeries safely, in areas of the brain and spine, that have been reserved to painful and time consuming “open” or “standard” procedures. According to non-limiting exemplary embodiments, it is also intended that the design modifications to the current retractor ports are to eliminate the use of dilating tubes, reducing the chance of prolapse and herniation of delicate human tissue into open space between said dilating tubes. Exemplary embodiments of the herein described access retractor ports are configured to accommodate ultrasound aspirators for use in and around vital structures of a body as described by PCT application PCT/US2015/027531.

### Advantageous Effects of Invention

The current invention is designed to modify and incorporate several of the common tools used in neurosurgery. Modifications to access equipment are vital to assure the safety and integrity of anatomy that is not essential to the surgery being performed. Combining navigation and monitoring technologies into the retractor systems will enable the surgeon to have continuous feedback of his/her location in the brain or spinal cord while actively performing the activities of surgery. By reducing the need to frequently change visual fields from microscope to computer screen, this will increase the operators' ability to perform said surgeries more efficiently. Further, the gasket reduces open spaces and improves preservation of tissues at least at distal ends of retractor tubes, dilating tubes and a locator rod.

### BRIEF DESCRIPTION OF THE DRAWINGS

FIG. 1 illustrates a retractor tube and gasket according to embodiments of the present invention.

FIG. 2 illustrates a series of dilating tubes having respective gasket fixation grooves according to embodiments of the present invention.

FIG. 3 illustrates a locator rod according to embodiments of the present invention.

FIG. 4a illustrates a top-down view of a retractor tube with integrated sensors according to embodiments of the present invention.

FIG. 4b illustrates a side-view of a retractor tube with integrated sensors according to embodiments of the present invention.

FIG. 5a illustrates a top-down view of an expandable retractor tube surrounded by an expandable sleeve.

FIG. 5b illustrates a side-view of an expandable retractor tube surrounded by an expandable sleeve.

FIG. 6 illustrates a flowchart of placing a retractor tube into a tissue.

FIG. 7 illustrates a flowchart of placing an expandable surgical retractor into a tissue.

### DESCRIPTION OF EMBODIMENTS

The advantages, features and aspects of the invention will become apparent from the following description of the embodiments with reference to the accompanying drawings, which is set forth hereinafter. Therefore, those skilled in the field of this art of the present invention can embody the technological concept and scope of the invention easily. In addition, if it is considered that detailed description on a related art may obscure the points of the present invention, the detailed description will not be provided herein. The specific embodiments of the present invention will be described in detail hereinafter with reference to the attached drawings.

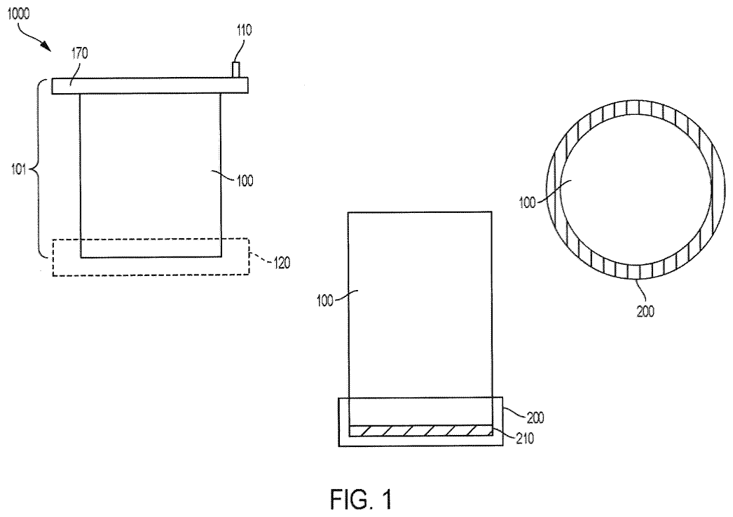

FIG. 1 illustrates a minimally invasive surgery (MIS) retractor tube with navigation fixation point and protective gasket 1000. The MIS retractor tube with navigation fixation point and protective gasket 1000 includes a retractor tube 100 and a gasket 200. Although gaskets are discussed herein, the gaskets may be interchanged with boots or expandable sleeves, as discussed further below.

The retractor tube 100 has a fixed length 101, navigation fixation point 110, and gasket fixation groove 210. The retractor tube 100 is configured to be inserted into a patient to provide 5 a view through an interior of the tube to a patient tissue, such as neural and/or surrounding tissues.

The retractor tube 100 may be composed of brain tissue compatible materials, such as stainless steel and/or titanium.

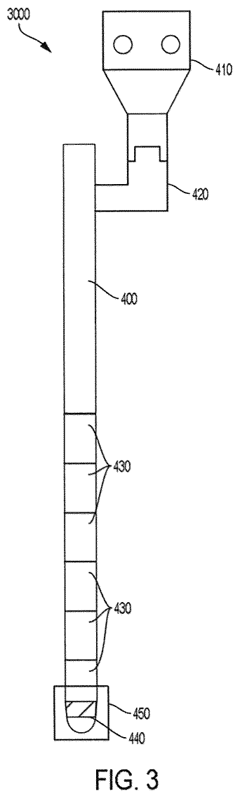

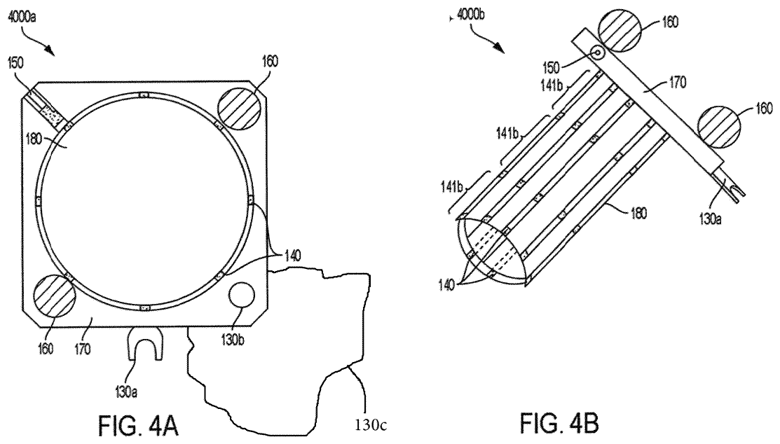

A proximal end of the retractor tube 100 includes a base plate 170 which may or may not be configured to attach to a patient's bone or other fixing structure 130c (representative of items to which the retractor fixing point 130b can be attached) to hold the retractor tube 100 in place, as will be described further in reference to the retractor fixation point 130a and retractor fixation point 130b of FIG. 4a. The retractor tube 100 also includes a navigation fixation point 110 to which a locator rod 400 may be attached as described further in reference to FIG. 3.

The retractor tube 100 is configured such that the gasket fixation groove 210 provide fixation points for a gasket 200 to be attached to a distal end 120 of the retractor tube 100. The gasket 200 provides increased friction and cushioning to a patient tissue, as opposed to a base retractor tube 100, and thereby prevents prolapse and herniation of tissue into the retractor tube 100 and/or unnecessary damage to the tissue. The gasket acts as a boot, sitting at an opening of the retractor tube 100.

The gasket 200 is configured to fit to the distal end 120 of the retractor tube 100 and to connect with the gasket fixation groove 210 thereof. According to exemplary embodiments, the gasket 200 fits to the distal end 120 according to any of elastic restoration of the gasket, mechanical interaction with the gasket fixation groove 210 and adhesive properties; however, this is merely exemplary and other equivalent means of fitting the gasket 200 to the retractor tube 100 may be employed.

The gasket 200 may be composes of any of silicone, latex, rubber and other soft, non-allergenic materials.

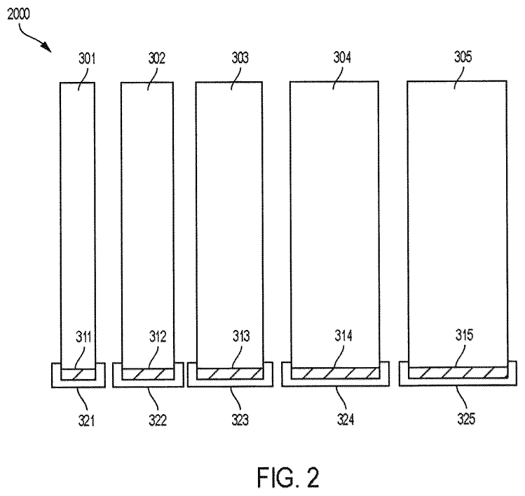

FIG. 2 illustrates MIS dilating tubes with gasket and gasket fixation grooves 2000. The MIS dilating tubes with gasket and gasket fixation grooves 2000 includes a sequence of dilating tubes 301-305.

The dilating tubes 301-305 are configured to be inserted into a patient to provide a sequence of expanding views to a patient tissue, such as neural and/or surrounding tissues. According to an example embodiment, diameters of the dilating tubes 301-305 increase from dilating tube 301 to dilating tube 305, and the dilating tube 301 is first inserted into a patient, and then either dilating tube 302 is inserted concentrically about dilating tube 301, or dilating tube 301 is removed and inserted into a different location. This process continues until dilating tube 305 is concentric about dilating tube 304 and or any of dilating tubes 301-303. The retractor tube 100 of FIG. 1 may then be inserted into the patient concentrically about dilating tube 305, as retractor tube 100 has a diameter greater than dilating tube 305.

As illustrated by FIG. 2, each of the dilating tubes 301-305 respectively has one of the gasket fixation grooves 311-315 at a distal end 120 thereof such that one of the gaskets 321-325 may be fixed thereupon as similarly described for the gasket 200 of FIG. 1. Each of the gasket fixation grooves 311-315 and gaskets 321-325 may have respective diameters so that the gaskets 321-325 may be fit to respective dilating tubes 301-305. The dilating tubes 301-305 create a channel for a dilating iris cylinder to be placed with gradual retraction of the patient's tissue and may have beveled edges.

FIG. 3 illustrates an MIS locator rod with gasket and gasket fixation grooves 3000. The MIS locator rod with gasket and gasket fixation grooves 3000 includes a locator rod 400.

The locator rod 400 includes a sensor fixation arm 420, fixed length increment lines 430, and gasket fixation groove 440 at a distal end thereof. The sensor fixation arm 420 of the locator rod 400 is configured such that a sensor 410, such as an infrared sensor, may be attached thereto. Hereinafter, the sensor 410 will be described as infrared sensor 410; however, this is merely exemplary and other sensors may be used for equivalent purposes; for example, the locator rod 400 may have other wired and wireless sensors attached thereto. The infrared sensor 410 provides data to a neuro-navigation computer (not-illustrated) to link the data about a patient's brain from the infrared sensor 410 to magnetic resonance imaging (MRI) images of the patient's brain. Such configuration allows for the infrared sensor 410 data visualization of tissue, such as a tumor, in real time correlated onto the MRI image. The fixed length increment lines 430 allow for a neuro navigational computer to calculate precise spatial points in conjunction with the data from the infrared sensor 410.

The locator rod 400 is seen separate on a neuro navigation computer and according to exemplary embodiments, is not affixed to a patient or table. The locator rod 400 may also be configured to incorporate the sensors as exemplarily discussed below.

The gasket fixation groove 440 of the locator rod 400 is configured such that a gasket 450 may be fit thereto similarly as described with respect to the gasket 200 and gasket fixation groove 210 of FIG. 1.

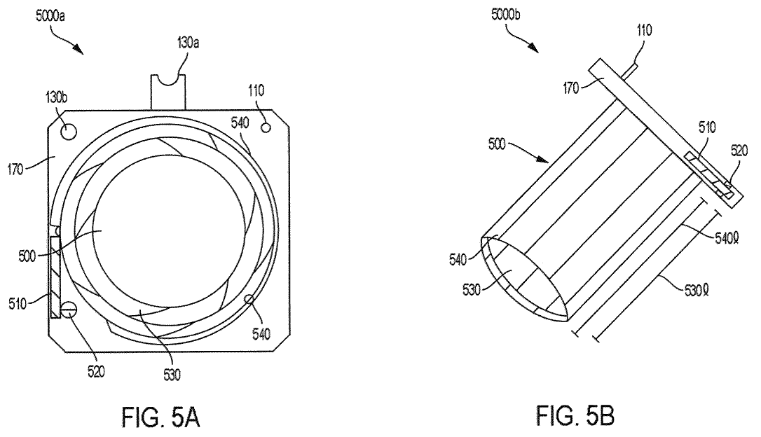

FIG. 4a illustrates an MIS retractor tube with integrated sensors 4000a. The MIS retractor tube with integrated sensors 4000a includes a retractor tube 180 with integrated neuro-monitoring points 140, neuro-monitoring receptacle 150 and reflector ball 160. The retractor tube 180 also includes a base plate 170 in which a retractor fixation point 130a and retractor 5 fixation point 130b are provided. The retractor tube 180 may be of fixed length, as exemplarily described for FIG. 1 or may be of expandable length as further described with respect to FIG. 5a.

The reflector ball 160 and the neuro-monitoring points 140, respectively incorporate sensors, of the retractor tube 180 provide data used by a neuro navigation computer to link the patient's brain to MRI images thereby allowing visualization of tissues, such as a tumor, real time correlated onto MRI images. The positions of the neuro-monitoring points 140 allow a neuro navigation computer to calculate precise spatial points. The neuro-monitoring points 140 are spaced about the circumference of the retractor tube 180 at equidistant intervals 142d.

The neuro-monitoring receptacle 150 provides data allowing for a surgeon to hear a loud tone, such as from compression to a nerve during surgery, and is a grounded system.

According to exemplary embodiments, the retractor fixation point 130a may be used to position the retractor tube 180 and the retractor fixation point 130b may be used to fix the base plate 170 of the retractor tube 180 to a patient tissue, such as a bone 130c (attaching hardware not shown).

FIG. 4b illustrates an MIS retractor tube with integrated sensors 4000b. The MIS retractor tube with integrated sensors 4000b includes the retractor tube 180 of FIG. 4a which includes the 20 retractor fixation point 130a, neuro-monitoring receptacle 150, reflector ball 160, and base plate 170. As illustrated in FIG. 4b, the neuro-monitoring points 140 are not only equidistantly located about respective circumferences of the retractor tube 180 but are also located at equidistant intervals 141d along the longitudinal length of the retractor tube 180 according to the exemplary embodiment of FIG. 4b. As discussed above, the position of the neuro-monitoring points 140 provide a compute with spatial information, such as a depth of the retractor.

FIG. 5a illustrates an MIS Retractor tube with sleeve 5000a. The MIS Retractor tube with sleeve 5000a includes expandable tube retractor 500 having a base plate 170 upon which a navigation fixation point 110, a retractor fixation point 130a, a retractor fixation point 130b, a threaded rod 510 and a screw 520 are mounted. The expandable tube retractor 500 also includes an expandable sleeve 540 surrounding an iris cylinder 530.

The expandable tube retractor 500 is configured such that interlocking veins of the iris cylinder 530 are actuated according to an action of the screw 520 and threaded rod 510 or other equivalent methods of actuation to expand or retract the iris cylinder 530. As the iris cylinder 530 expands, so does the expandable sleeve 540 which covers an exterior of the expandable sleeve 540.

FIG. 5b illustrates an MIS Retractor tube with sleeve 5000b. The MIS Retractor tube with sleeve 5000b illustrates that the expandable sleeve 540 covers an exterior of the iris cylinder 530. The expandable sleeve 540 improves the friction and cushioning of the expandable tube retractor 500 to protect and preserve patient tissue and also to prevent prolapse and herniation of patient tissue into the expandable tube retractor 500. According to exemplary embodiments, the length 5041 of the expandable sleeve 540 is greater than the length 5301 of the iris cylinder 530.

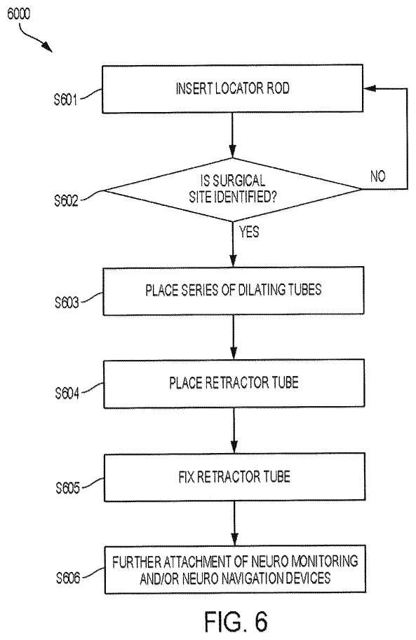

FIG. 6 illustrates a flowchart 6000 of placing a retractor tube, such as the retractor tube 100 of FIG. 1.

At S601, a locator rod, such as locator rod 400, is inserted into a human tissue such as during brain or spinal surgery. The locator rod may have a gasket attached to a distal end thereof.

At S602, the data retrieved from the locator rod is used to identify if the locator rod has been inserted at a desired a surgical site. If not, the locator rod is reinserted into a different location of tissue.

At S603, a series of dilating tubes are placed at the desired surgical site, such as described for the dilating tubes 301-305. The dilating tubes may have respective gaskets attached to distal ends thereof.

At S604, a retractor tube, such as retractor tube 100 is placed into the tissue dilated by the series of dilating tubes. The retractor tube may have a gasket attached to a distal end thereof.

At S605, the retractor tube is fixed. For example a baseplate of the retractor tube may be fixed to a boney structure such as a skull during brain surgery or to an attachment arm that is securable to an operating room bed 130c (attaching hardware not shown).

At S606, further neuro monitoring and/or neuro navigation devices are attached to the retractor tube.

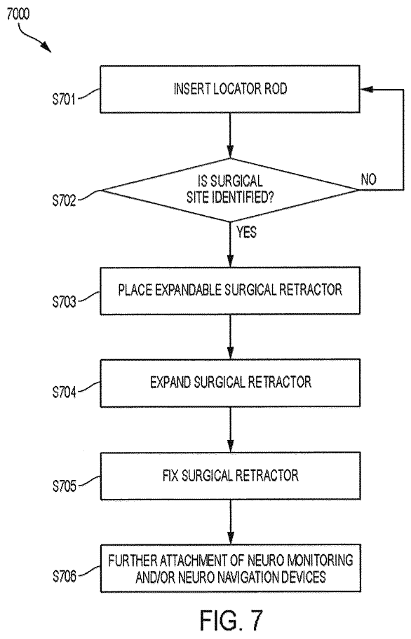

FIG. 7 illustrates a flowchart 7000 of placing an expandable surgical retractor, such as the expandable tube retractor 500 of FIG. 5a.

At S701, a locator rod, such as locator rod 400, is inserted into a human tissue such as during brain or spinal surgery. The locator rod may have a gasket attached to a distal end thereof.

At S702, the data retrieved from the locator rod is used to identify if the locator rod has been inserted at a desired a surgical site. If not, the locator rod is reinserted into a different location of tissue.

At S703, an expandable retractor is placed at the desired surgical site. The expandable retractor may have an expandable sleeve attached to an exterior of an expandable retractor tube of the expandable retractor.

At S704, the expandable retractor is expanded as is the expandable sleeve.

At S705, the expandable retractor is fixed. For example a baseplate of the expandable retractor may be fixed to a boney structure such as a skull during brain surgery or to an attachment arm that is securable to an operating room bed.

At S706, further neuro monitoring and/or neuro navigation devices are attached to the expandable retractor.

While the present invention has been described with respect to certain preferred embodiments, it will be apparent to those skilled in the art that various changes and modifications may be made without departing from the scope of the invention as defined in the following claims.

### INDUSTRIAL APPLICABILITY

The negative ramifications to these design modifications are the potential of harming the patient with active use. However, this is by nature an acceptable risk that the patient incurs with consent to an operation.

According to the CDC, in the United States, the proportion of the population aged >65 years is projected to increase from 12.4% in 2000 to 19.6% in 2030. The number of persons aged >65 years is expected to increase from approximately 35 million in 2000 to an estimated 71 million in 2030, and the number of persons aged >80 years is expected to increase from 9.3 million in 2000 to 19.5 million in 2030. The increased number of persons aged >65 years will potentially lead to increased health-care costs. The health-care cost per capita for persons aged >65 years in the United States and other developed countries is three to five times greater than the cost for persons aged <65 years, and the rapid growth in the number of older persons, coupled with continued advances in medical technology, is expected to create upward pressure on health- and long-term-care spending. With the projected growth of the “Baby Boomer” population, the need for surgical intervention is expected to grow. By reducing post-surgical recovery time, surgery time and potential injury exposure, this can lead to cost savings both in healthcare dollars and litigation expenses.

### REFERENCE SIGNS LIST

100 RETRACTOR TUBE
101 FIXED LENGTH
110 NAVIGATION FIXATION POINT
120 DISTAL END
130a RETRACTOR FIXATION POINT
130b RETRACTOR FIXATION POINT
140 NEURO-MONITORING POINTS
141d INTERVALS
142d INTERVALS
150 NEURO-MONITORING RECEPTACLE
160 REFLECTOR BALL
170 BASE PLATE
180 RETRACTOR TUBE
200 GASKET
210 GASKET FIXATION GROOVE
301-305 DILATING TUBES
311-315 GASKET FIXATION GROOVES
321-325 GASKETS
400 LOCATOR ROD
410 SENSOR
420 SENSOR FIXATION ARM
430 FIXED LENGTH INCREMENT LINES
440 GASKET FIXATION GROOVE
450 GASKET
500 EXPANDABLE TUBE RETRACTOR
510 THREADED ROD
520 SCREW
530 IRIS CYLINDER
5301 LENGTH
540 EXPANDABLE SLEEVE
5401 LENGTH
1000 MIS RETRACTOR TUBE WITH NAVIGATION FIXATION POINT AND PROTECTIVE GASKET
2000 MIS DILATING TUBES WITH GASKET AND GASKET FIXATION GROOVES
3000 MIS LOCATOR ROD WITH GASKET AND GASKET FIXATION GROOVES
4000a, 4000b MIS RETRACTOR TUBE WITH INTEGRATED SENSORS
5000a, 5000b MIS RETRACTOR TUBE WITH SLEEVE
6000 METHOD OF PLACING RETRACTOR TUBE
7000 METHOD OF PLACING AN EXPANDABLE SURGICAL RETRACTOR

### CITATION LIST

US20070208229 A1
U.S. Pat. No. 8,303,497 B2
US20130006059 A1

What is claimed:

1. A retractor tube comprising:
a distal end configured to incorporate any of a gasket, a boot or an expandable sleeve; and
a base plate, at a proximal end of the retractor tube, configured to secure the retractor tube to any of an operating room table, a patient fixation device and a boney structure of a patient.

2. The retractor tube according to claim 1, wherein the base plate is further configured to fix a neuro navigation infrared sensor thereto.

3. The retractor tube according to claim 1, wherein the base plate is further configured to incorporate any of wirings and wireless ports for neuro monitoring devices and sensors.

4. The retractor tube according to claim 1, further comprising:
a plurality of sensors integrally provided throughout both a length and a circumference of the retractor tube.

5. The retractor tube according to claim 1, wherein the retractor tube is configured to be an expandable diameter tube comprising an expandable iris cylinder with interlocking veins.

6. The retractor tube according to claim 5, wherein the expandable iris cylinder is actuated according to any of a threaded rod, a screw and a rotating arm.

7. The retractor tube according to claim 1, wherein
either one of the gasket or the expandable sleeve comprises any of expandable silicone, rubber, latex or other hypo-allergenic material.

8. A cylindrical sleeve configured to cover and expand with an outer side of an expandable iris cylinder of a retractor tube.

9. The cylindrical sleeve of claim 8, wherein the cylindrical sleeve comprises any of expandable silicone, rubber, latex or other hypo-allergenic material.

10. A locator rod comprising:
a proximal end configured to incorporate neuro navigation and neuro monitoring sensors.

11. The locator rod according to claim 10 further comprising:
an attachment arm configured to fix any of wired and wireless sensors thereto, wherein
the locator rod is configured to perform neuro monitoring and neuro navigation.

12. The locator rod according to claim 10, further comprising a distal end configured to incorporate a gasket comprising any of expandable silicone, rubber, latex or other hypo-allergenic material.

13. A series of dilating tubes respectively comprising:
a distal end configured to accommodate expansion of any of a gasket or boot.

14. The series of dilating tubes according to claim 13, wherein
the gasket or boot comprises any of expandable silicone, rubber, latex or other hypoallergenic
material.

15. A gasket, wherein
the gasket is configured to attach to at least any distal end of a retractor, a dilating tube and a locator rod.

16. The gasket according to claim 15, wherein
the gasket comprises any of expandable silicone, rubber, latex or other hypo-allergenic material.

17. A method of retracting human tissue during brain or spinal surgery, the method comprising:
inserting a locator rod with neuro monitoring and neuro navigation capabilities to identify a desired surgical site.

18. The method of claim 17, further comprising:
placing, at the desired surgical site, a series of dilating tubes with respective gaskets affixed to outer, distal ends of each tube.

19. The method of claim 18, further comprising:
placing, at the desired surgical site, a surgical retractor tube comprising a base plate
configured to fix the surgical retractor tube, to any of a boney structure or an attachment arm securable to an operating room bed; and attaching any of neuro monitoring and neuro navigation devices to the base plate.

20. The method of claim 17, further comprising:
placing, at the desired surgical site, an expandable surgical retractor tube comprising a base plate configured to fix the expandable surgical retractor tube, to any of a boney structure or an attachment arm securable to an operating room bed; and attaching any of neuro monitoring and neuro navigation devices to the base plate.
